(19) United States
(12) Patent Application Publication
(10) Pub. No.: US 2017/0131234 A1
(43) Pub. Date: May 11, 2017

(54) Gel Electrophoresis and Transfer Combination using Conductive Polymers and Method of Use

(71) Applicant: Woodham Biotechnology Holdings, LLC, Beverly Hills, CA (US)

(72) Inventor: Andrew Woodham, Los Angeles, CA (US)

(73) Assignee: Woodham Biotechnology Holdings, LLC, Beverly Hills, CA (US)

(21) Appl. No.: 15/017,540

(22) Filed: Feb. 5, 2016

Related U.S. Application Data
(60) Provisional application No. 62/253,143, filed on Nov. 10, 2015.

(51) Int. Cl. G01N 27/447; G01N 33/531

(52) U.S. Cl. CPC G01N 27/44739; G01N 33/531; G01N 27/44756; G01N 27/44708; G01N 27/44713

(57) ABSTRACT

A precast gel/membrane combination unit for use in gel electrophoresis and membrane transfer for protein analysis, and method of use. Transparent electrically conductive plastic/polymer plate(s) house an electrophoresis gel and immunoblotting membrane. The gel and membrane are positioned between two plates of conductive plastics/polymers or plates having an electrically conductive layer or film. During the electrophoresis step, electrical current moves proteins through the gel allowing for protein separation. Then, without removing or reorienting the gel or apparatus, the electrical contacts are switched to allow the flow of electricity to run perpendicularly through the gel via the conductive plastics/polymers, which will allow the proteins to transfer to the membrane housed in the same precast gel/membrane combination unit. The apparatus allows the user to visually see and perform the steps of protein separation and protein transfer to a transfer membrane without transferring the gel or apparatus after the electrophoresis separation phase.

### CROSS REFERENCE TO RELATED APPLICATIONS

This application claims the benefit of U.S. Provisional Patent Application No. 62/253,143, filed on Nov. 10, 2015, the entirety of which is hereby incorporated by reference.

### FIELD OF THE INVENTION

The present invention relates generally to gel electrophoresis and transfer with one precast gel/membrane combination using conductive plastics/polymers.

### BACKGROUND OF THE INVENTION

Although western blotting is a common technique, there are still many issues that arise from the transferring step. These include the introduction of air bubbles when placing gels on membranes, and as gels have become thinner to reduce the amount of protein needed, the ripping or tearing of gels as they are moved from a precast setting to the transfer membrane has become more problematic. These complications can be devastating when the analysis of limited amounts of protein is required, especially when no more protein is available or it is a clinical specimen. Furthermore, the concept of a protein separation/transfer combo is hindered by the current use of insulating plastics to house precast gels. Additionally, researchers often prefer to watch the electrophoresis during progress, which limits the use of non-transparent materials such as metals for run/transfer combinations.

It would be desirable to have a one-step separation and transfer western blot method that would utilize a single precast gel and membrane combination. While western blotting is a fundamental technique, there are still many issues that arise from the step of transferring the proteins separated via gel electrophoresis to a probe-able membrane such as the introduction of air bubbles and gel ripping or tearing when placing gels on membranes. Hence, it would be advantageous to have a means to avoid the introduction of air bubbles, and to avoid the ripping or tearing of gels. Therefore, there currently exists a need in the industry for a device and associated method that can perform gel electrophoresis and membrane transfer in one precast gel/membrane combination. Additionally, researchers prefer to watch the electrophoresis steps during progress, and hence the use of clear conductive plastics/polymers can meet these needs. Therefore, the use of conductive plastics/polymers with particular resistivity in western blotting applications solves fundamental problems associated with current methods.

Conductive plastics/polymers are electrically conductive materials that can be shaped and molded into hardened structures made from organic or synthetic polymers such as, but not limited to: polyacetylene, poly(pyrrole)s (PPY), polyanilines, poly(thiophene)s (PT), poly(3,4-ethylenedioxythiophene) (PEDOT), poly(p-phenylene sulfide (PPS), polyactylene)s (PAC), poly(p-phenylene vinlene) (PPV), and their derivatives.

There have been many attempts to simplify the separation and transfer of proteins or other macromolecules using various apparatuses and techniques. U.S. Pat. No. 4,994,166 to Fernwood et al. describes a single apparatus for slab gel electrophoresis and blotting, both of which are performed in a single tank cell, which contains separation electrodes along opposing vertical walls and blotting electrodes arranged horizontally above and below the level of gel placement. The cell is operated in separatory and blotting modes, in which separatory and blotting electrodes are separately energized. No means for separation of gel and blotting membrane are provided. Fernwood requires porous gel supports to allow the electric field to pass through the membrane and the top plate transfer electrode must be removed from contact with the buffer solution during the separatory phase.

U.S. Pat. No. 5,102,524 to Dutertre describes a multiple electrophoresis method, where different sets of electrodes are used in a two-step process to first separate macromolecules and then to transfer them to a deposition membrane.

U.S. Pat. No. 5,593,561 to Cognard shows a multiple electrophoresis method for controlled migration of macromolecules and transfer thereof to a membrane in a vessel, containing a plurality of parallel elongate electrodes. The first electric field, established between electrodes, provides means for macromolecular separation in a gel, and the second electric field, perpendicular to the first, provides means for transferring the macromolecules onto the membranes. In the described method, at first, electrodes and transfer membranes are assembled in the vessel, which is then filled with gel. After the separation of macromolecules in a gel and transfer to membranes, gel is liquefied, dissolved, or decomposed, allowing the removal of membranes. The invention is for use without prefabricated gel-membrane units.

U.S. Pat. No. 8,173,002 to Margalit discloses a dry blotting system to transfer proteins onto a transfer membrane. The system does not include an electrophoresis device, so the device does not allow the user to visualize the separation and blotting in a single device. The device requires the user to be present to transfer the gel to a transfer membrane on the blotting device. Margalit teaches the use of electrically conducting polymers, but not in combination with a single device that both separates proteins and transfers the proteins to a transfer membrane. Margalit does not teach the use of any transparent electrically conducting polymers and the polymers are not part of a gel-supporting cassette.

U.S. Patent Appl. Pub. No. 2006/0042951 to Ohse discloses an apparatus to separate and transfer proteins via the use of a fine grove, a transferring electrode and a transparent conductive material having a thickness of approximately 0.1 μm. The apparatus includes a pair of separating electrodes for causing a substance in a sample to move along a passage, and a pair of transferring electrodes for causing the substance in the sample to be transferred to the capturing material by electrophoresis. The transparent conductive material is not capable of being the support structure due to its thickness of approximately 0.1 μm, which would not have sufficient strength to serve as the supporting walls for a gel. The separation and blotting is performed in an electrophoresis buffer and does not make use of a gel slab or gel slab assembly, which are commonly used for western blots.

U.S. Pat. No. 6,602,391 to Serikov discloses an apparatus and method for capillary separation of macromolecules and post-separation blotting. However, Serikov does not disclose the use of a slab gel where the user can view the separation of macromolecules and transfer the macromolecule to a blotting membrane for western blotting.

Conductive polymers have previously been described, but not in conjunction with electrophoresis and blotting. Ates et al. described numerous applications of conducting polymers in “Conducting Polymers and their Applications” (Current Physical Chemistry, 2012, 2, 224-240). International Patent Application No. PCT/EP2013/065163 to Jung discloses a conductive polymer composition and transparent electrode for an antistatic layer. International Patent Application No. PCT/KR2008/002236 to Kim discloses a conductive polymer for use as a transparent electrode and the method of fabricating the electrode using an ink jet spray method. U.S. patent application Ser. No. 13/616,804 discloses a transparent panel and method of manufacturing a transparent panel where a conductive polymer layer is formed to make a transparent electrode.

Transparent conductive plates using conductive polymers have not been used in electrophoresis and blotting apparatuses where the conductive plates are used as the gel support for creation of a pre-cast gel so that the pre-cast gel and its conductive polymer housing can be used for both electrophoresis and blotting without removing the gel after electrophoresis separation to thereafter blot the proteins on a transfer membrane. Although there are some metal compositions that are transparent, such as indium tin oxide, which is a transparent metal composition and has been used in some applications where both conductivity and transparency are required, a considerable compromise must be made between conductivity and transparency. In transparent metal compositions, increasing the thickness and increasing the concentration of charge carriers increase the material's conductivity, but dramatically decrease its transparency. Thus, a thin film of indium tin oxide is both transparent and conductive, but when thickness and rigidity are both required (such as in plates used to create and support a pre-cast gel), then the conductive transparent metals are no longer transparent.

All patents, patent applications, and non-patent applications disclosed in the background and description of the invention are hereby incorporated by reference for all purposes in their entireties.

### SUMMARY OF THE INVENTION

The present invention advantageously fills the aforementioned deficiencies by providing gel electrophoresis and transfer with one precast gel/membrane combination using conductive plastics/polymers, which provides a fast, reliable, and easy method to perform a hands-free protein separation followed by an efficient transfer.

The invention includes a method for gel electrophoresis and membrane transfer in a precast gel/membrane combination. The precast gel/membrane combination consists of conductive plastic/polymer casings, electrophoresis gels, and western blot membranes. The gel and membrane pair is sandwiched between two sheets of the conductive plastic/polymer.

The present invention may also have one or more of the following: a thin layer of a less conductive gel (i.e. high percentage polyacrylamide) between the gel and the membrane; different types of electrophoresis gels including those made from polyacrylamide, bis-Tris, Tris-acetate, etc.; different immunoblotting membranes including those made from nitrocellulose, and polyvinylidene difluoride (PVDF); different conductive plastic/polymer materials; plastic insulators; a buffer tank and buffer lid; precast gel/membrane holder cassette with electrodes; negative electrode chamber; positive electrode chamber; electrode assembly; anode and cathode buffers; cooling unit; and a programmable power source.

The present invention is unique in that it utilizes innovative conductive plastics/polymers with particular resistivity in western blotting applications to solve fundamental problems in current methods, and that allow for convenient, one-step electrophoresis/transfer methods.

The present invention is unique in that it is different from other known processes or solutions. More specifically, the present invention owes its uniqueness to the fact that it utilizes conductive plastics/polymers to house precast gel/membrane combinations that can act as an insulator in one scenario and an electrode in another scenario, which is advantageous for a device that separates proteins in one direction using one pair of electrodes and transfers proteins in a perpendicular manner to a blotting membrane through the use of a different pair of electrodes.

It is an object of the present invention to provide gel electrophoresis and transfer with one precast gel/membrane combination using conductive plastics/polymers that does not suffer from any of the problems or deficiencies associated with prior solutions.

In one embodiment there is an apparatus for electrophoretic separation and blotting. The apparatus has a first electrically conductive plate made from a transparent conductive polymer and a second electrically conductive plate substantially parallel to the first electrically conductive plate. The apparatus has an electrophoresis gel and a blotting membrane where the electrophoresis gel is located between the first electrically conductive plate and the blotting membrane. The blotting membrane is between the electrophoresis gel and the second electrically conductive plate.

In another embodiment, the apparatus also includes a low conductivity (high resistivity) gel between the second electrically conductive plate and the blotting membrane. The embodiment also includes filter paper between the second conductive plate and the blotting membrane.

In yet another embodiment the first transparent electrically conductive plate has electrically conducting wires arranged in an array or grid to disperse current/charge.

In yet another embodiment, the first plate is generally formed from a non-electrically conductive static-dissipative material, but has a thin electrically conducting polymer layer or thin electrically conducting film disposed on the first plate's inner surface to act as a plate electrode during the blotting phase.

In yet another embodiment, the apparatus includes a liquid receptacle tank having an upper buffer chamber and lower buffer chamber each with a separation phase electrode. The tank also has a pair of blotting phase electrodes arrange in a manner so that the electric field produced from the separation phase electrodes is substantially perpendicular to the electric field produced from the blotting phase electrodes. The apparatus also includes a power supply configured to automatically or manually switch between, and apply, a voltage to the separation phase electrodes and a voltage to the blotting phase electrodes.

In yet another embodiment, there is a method for separation and post-separation blotting of macromolecules to a blotting membrane. The user provides an apparatus in a first orientation within a liquid receptacle tank. The apparatus has a first electrically conductive plate composed of a transparent conductive polymer, a second electrically conductive plate substantially parallel to the first electrically conductive plate, an electrophoresis gel, and a blotting membrane. The electrophoresis gel is located between the first conductive plate and the blotting membrane, and the blotting membrane is located between the electrophoresis gel and the second electrically conductive plate. The user separates the macromolecules (e.g. proteins) along the gel of the apparatus by applying a first electrical driving force to a pair of separation electrodes. This step occurs while the gel and transfer membrane are in a first orientation. The electrical force applied to the separation electrodes is then discontinued. Without removing the gel and transfer membrane from the liquid receptacle tank, and also while maintaining the orientation of the gel and membrane in the liquid receptacle tank, a second electrical driving force is applied to a pair of blotting electrodes substantially perpendicular to the first electrical driving force. The orientation of the gel/membrane unit is maintained relative to both the separation and blotting electrodes.

The present invention now will be described more fully hereinafter with reference to the accompanying drawings, which are intended to be read in conjunction with both this summary, the detailed description, and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete, and will fully convey the full scope of the invention to those skilled in the art.

### BRIEF DESCRIPTION OF THE DRAWINGS

These and other features and advantages of the present invention will become appreciated as the same becomes better understood with reference to the specification, claims, and drawings herein:

FIG. 1 shows a side view of the general setup of the precast gel/membrane combination unit for electrophoresis and transfer;

FIG. 2 shows a front view of a typical precast gel/membrane combination typically used for electrophoresis as known in the prior art;

FIG. 3 shows a cross sectional side view of the of the precast gel/membrane combination unit within an electrophoresis and transfer tank;

FIG. 4 shows a perspective view of the electrophoresis and transfer tank without the precast gel/membrane combination unit placed inside the tank;

FIG. 5 shows a front view of an embodiment of the precast gel/membrane combination unit;

FIG. 6 shows a side view of an embodiment of the gel/membrane combination unit having a conductive wire mesh and thin conductive polymer or film in contact with electrophoresis gel;

FIG. 7 shows a perspective view of an embodiment of the gel/membrane combination unit;

FIG. 8 shows a top view of an embodiment of the gel/membrane combination unit having projections on one plate to create a gap for the gel and membrane.

FIG. 9 shows a perspective view of the embodiment of FIG. 8.

### DETAILED DESCRIPTION OF THE DRAWINGS

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present therebetween. As used herein, the term “and/or” includes any and all combinations of one or more of the associated listed items.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, and/or section from another element, component, region, layer, and/or section.

It will be understood that the elements, components, regions, layers and sections depicted in the figures are not necessarily drawn to scale.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom,” “upper” or “top,” “left” or “right,” may be used herein to describe one element's relationship to another element(s) as illustrated in the Figures. It will be understood that relative terms are intended to encompass different orientations of the device in addition to the orientation depicted in the Figures.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing. The invention illustratively disclosed herein suitably may be practiced in the absence of any elements that are not specifically disclosed herein.

The present invention is directed to gel electrophoresis and transfer with one precast gel/membrane combination unit 10 using conductive plastics/polymers.

Electrophoresis may be performed using a variety of methods, including but not limited to sodium dodecyl sulfate polyacrylamide gel electrophoresis (SDS-PAGE).

In one embodiment, the invention is made of the following components: a precast gel/membrane combination unit 10 that includes the gel 6 and transfer/blotting membrane 12 sandwiched between two sheets of conductive plastics/polymers 2, 4. Additionally, a gel having low conductivity 8 separates the gel 6 and membrane 12.

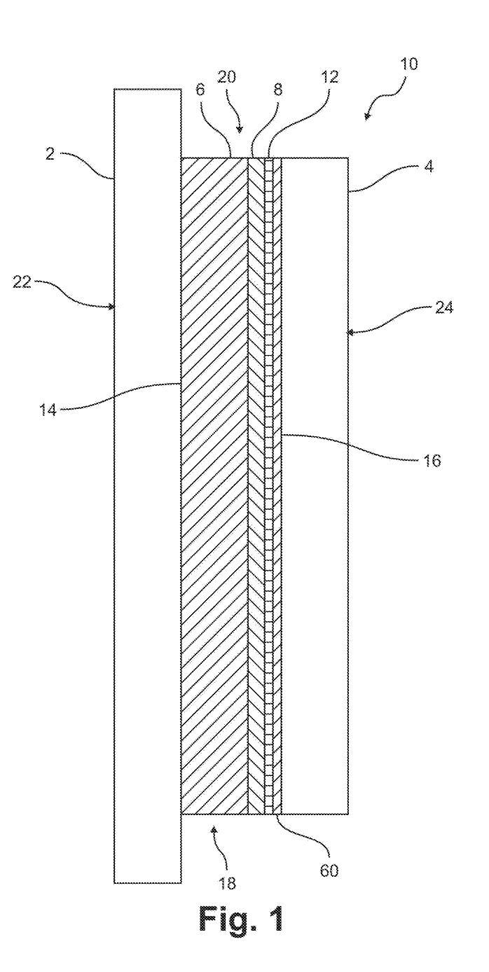

FIG. 1 shows a side view of one embodiment of the general setup of the precast gel/membrane combination unit 10 for electrophoresis and transfer. Generally, a gel 6 and membrane 12 are sandwiched between two sheets of conductive plastics/polymers 2, 4. Additionally, the gel 6 and membrane 12 may be separated by a small layer of a low conductive (i.e. high percentage polyacrylamide) gel 8. During separation mode, current flows from the upper surface 20 of the gel 6 to the lower surface 18 of the gel 6. During blotting mode (i.e. protein transfer mode), current flows from the first conductive plate 2 to the second conductive plate 4.

The first conductive plate 2 is made from a transparent conductive material, such as polyanilines, polypryrrols, polythiophenes, or other transparent conductive polymer. The first conductive plate has an outer surface 20 and inner surface 14. The second conductive plate 4 is also made from a conductive material, but need not be transparent. The advantage in using a system that includes a transparent conductive material instead of a non-transparent conductive material is that users often prefer to watch the separation phase of electrophoresis in order to visually determine the extent of protein movement/separation during electrophoresis. Typical plate electrodes are metal and therefore non-transparent. If typical metal plate electrodes are used along the surface of a gel, users cannot determine to what extent proteins have separated during electrophoresis. In one embodiment, only a single conductive plate needs to be transparent for the user to visually determine how much protein movement has occurred because protein movement can be observed by looking at one side of a gel. The second side of the gel 6 will be blocked from view by the transfer/blotting membrane. In a preferred embodiment, the polymer used for the conductive plate 2 has a volume resistivity in the range of 10³-10⁵ ohm-cm, but may be as high as 10⁸ ohm-cm.

Generally, the polymers used to create support structures for electrophoresis gels are polymers, and therefore electrically insulating. However, there is a special class of polymers that intrinsically conduct electricity at levels much higher than semiconductors (up to 1000 S/cm), and their conductivities/resistivities can be controlled through different methods of production. Conductive polymers are organic polymers that conduct electricity. Specifically, they offer electrical conductivity less than metals, and can have properties of plastics, such as transparency. The electrical properties (i.e. resistivity) can be fine-tuned using organic synthesis methods and dispersion techniques. Types of organic conductive polymers include polyacetylene, poly(pyrrole)s (PPY), polyanilines, poly(thiophene)s (PT), poly(3,4-ethylenedioxythiophene) (PEDOT), poly(p-phenylene sulfide (PPS), poly(acetylene)s (PAC), poly(p-phenylene vinylene) (PPV), and their derivatives. Conductive polymers may be made from combinations of conductive polymers or combinations of derivatives of the polymers. Generally, the electrical conductivity of a polymer is created by removing an electron from the polymer's conjugated π-orbital via doping and the delocalization of electrons along the polymer backbone.

To ensure that an electric field is evenly distributed along the entirety of the conductive plates, the composition of the plates should have high static-dissipative properties. To ensure a substantially equal electric field emanating from all regions of each conductive plate, the outer surface of a conductive plate may have one or more thin wires (or nanowires) disposed on its outer surface. The wires may be arranged in an array or grid-like shape or mesh. The wires are unobtrusive so that they do not prevent the user from being able to see the gel through the wires and conductive plate in order to allow the user to monitor protein separation during electrophoresis. Preferred embodiments having wires or grids of wires spaced between 0.5 cm and 1.0 cm apart may be sufficient to create a plate electrode having a substantially even electric field emanating from its surface.

Referring again to FIG. 1, adjacent to the inner surface 14 of the first conductive plate 2 is an electrophoresis gel 6. The electrophoresis gel 6 is a typical slab gel. The gel 6 may be made from any number of compositions known in the art, including agarose, polyacrylamide, Tris-glycine, bis-Tris, and Tris-acetate. Agarose gels would typically be used for DNA and RNA analysis and polyacrylamide gels, Tris-glycine, bis-Tris, Tris-acetate for protein analysis. Typical resolving gels for protein analysis are made between 6% and 15% polyacrylamide. In preferred embodiments, a bis-Tris gel is in a range of 10% to 12% and a Tris-acetate gel is in a range of 7%-10%, but values may lie outside these ranges depending on the size of the protein that one wishes to analyze or probe in the sample. For example, the smaller the known weight of a macromolecule, the higher the percentage of gel should be used. The dimensions of the electrophoresis gel 6 are typically rectangular and in a preferred embodiment are approximately 10 cm×10 cm, but may vary depending on the number of samples to be run simultaneously, the type of sample, and the sample volume. In a preferred embodiment, the precast gel/membrane combination unit 10 is less than 1 cm thick, but may also be designed thicker. On the opposing side of the electrophoresis gel 6 is a low conductivity (i.e. high percentage) gel 8.

Adjacent to the low conductivity gel 8 is a transfer membrane 12. The transfer membrane, also known as an immobilization membrane, may be of any of a wide range of blotting materials, such as blotting paper, nitrocellulose, PVDF, nylon, and other materials, as well as such materials in treated or derivatized form, as well known among those skilled in the art. The use of the membrane 12, and method by which the macromolecules are transferred from the electrophoresis gel 6 through the low conductivity gel 8 to the membrane 12 is discussed in further detail below. The low conductivity gel 8 between the transfer membrane 12 and electrophoresis gel 6 prevents the direct contact of the transfer membrane 12 with the electrophoresis gel 6 during electrophoresis. Since proteins have a high affinity to western blot transfer membranes 12, the low conductivity gel 8 prevents proteins from binding to the surface of the membrane 12 during electrophoresis.

Adjacent to the transfer membrane 12 is filter paper 60. The filter paper 60 is sandwiched between the high conductive gel 8 and second conductive plate 4. Filter paper 60, when wet, acts as an ion reservoir, thereby aiding in the transfer of macromolecules to the membrane 12. Filter paper also ensures that the transfer membrane 12 stays wet. The transfer membrane 12 and filter paper 60 may be pre-wet prior to assembly of the gel/membrane combination unit 10 with a methanol solution, other wetting buffer, or the filter paper 60 may be wet from the buffer solution used in the electrophoresis and blotting phases. A transfer membrane-wetting buffer typically includes methanol.

The gel/membrane combination unit 10 of FIG. 1 includes the electrophoresis gel 6, low conductivity gel 8, transfer membrane 12, and filter paper 60, all sandwiched between the transparent first conductive plate 2 and second conductive plate 4. Embodiments without the low conductivity gel 8 and/or filter paper 60 may also serve to both separate macromolecules and transfer the macromolecules to the transfer membrane 12.

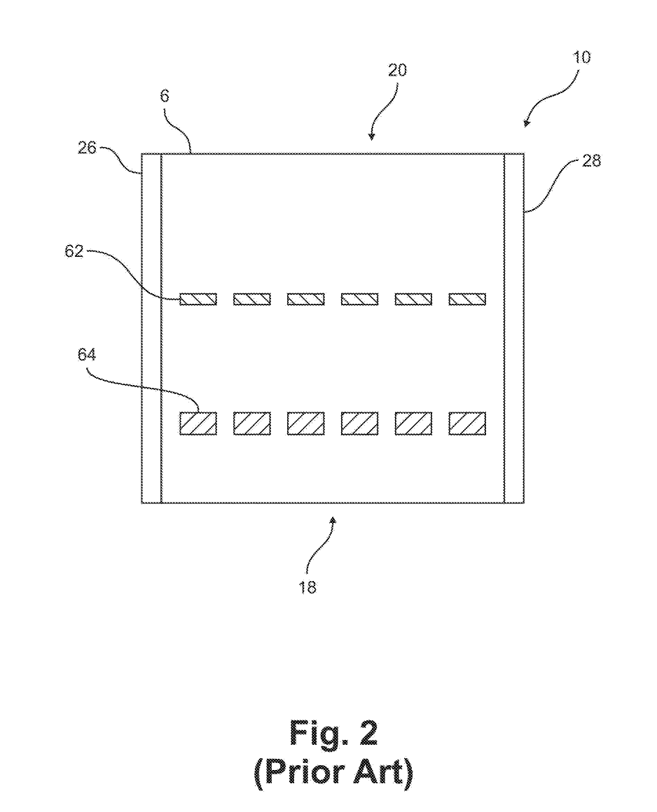

FIG. 2 shows the front view of the precast gel/membrane combination unit 10 for electrophoresis and transfer. During the electrophoresis separation phase, proteins move vertically down from the top of the gel 20 to the bottom of the gel 18. During electrophoresis, larger proteins (shown as upper bands 62) move slower through the gel 6 than smaller proteins (shown as lower bands 64). In one embodiment, there are two non-conductive insulative plastic strips 26, 28 that flank the sides of the gel 6, but that are also sandwiched between the conductive plastic/polymer sheets 2, 4 to direct current through the gel 6 during the electrophoresis step. These strips also provide structural support and rigidity to the cassette. The movement of the proteins during the transfer/blotting step is along the z-axis, perpendicular to the to the direction of protein separation along the y-axis.

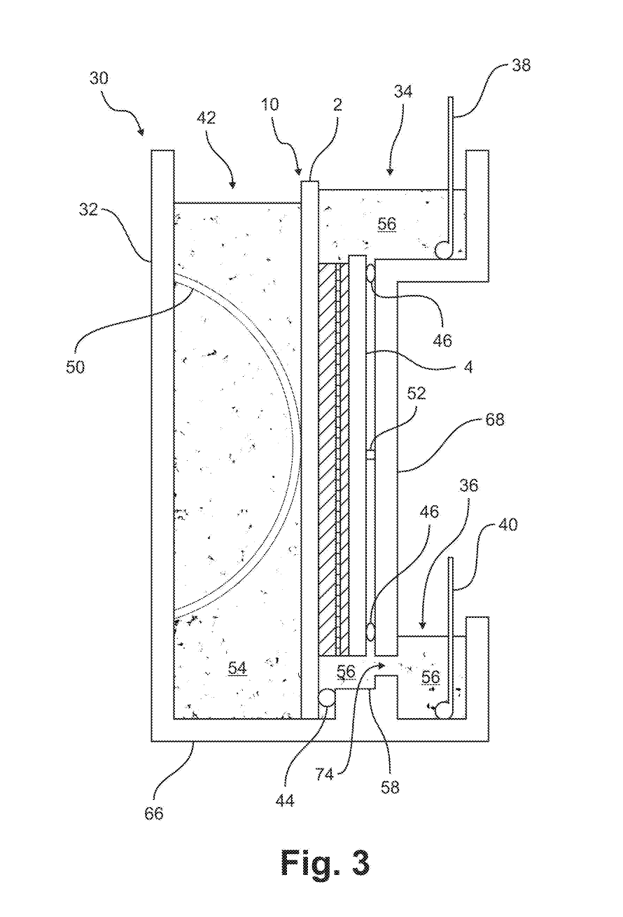

The tank apparatus 30 of FIG. 3, which together with the precast gel/membrane combination unit 10, serves as the system for both the electrophoresis separation phase and blotting phase. The tank apparatus 30 is a liquid receptacle that includes a front panel 32, rear panel 68, first side panel 70, second side panel 72, bottom panel 66, lip 58 on the rear panel 68, and a lid (not shown). The lip 58 may be a variety of shapes but in a preferred embodiment is substantially U-shaped along the inner walls of the first and second side panels 70, 72 of the tank apparatus 30.

Gels for protein separation and transfer are usually submerged in an electrolyte containing buffered solution such as Tris-acetate-EDTA (TAE) buffer in a tank apparatus 30. Other buffers may be used depending on the type of gel used in the precast gel/membrane combination unit 10. For example, a Tris-acetate buffer may be used for Tris-acetate gels, whereas 2-(N-morpholino)ethanesulfonic acid (MES) or 3-(N-morpholino)propanesulfonic acid (MOPS) buffers may be used for bis-Tris gels. The buffers in this system should be efficient for both the electrophoresis phase and transfer phase. The tank apparatus 30 has an upper chamber 34 and a lower chamber 36. A first separation phase negative electrode (cathode) 38 is disposed within the upper chamber 34. A second separation phase positive electrode (anode) 40 is disposed within the lower chamber 36. The first and second separation phase electrodes 38, 40 are each connected to a programmable power source (not depicted) to power the separation electrodes 38, 40. Power sources for tank apparatuses for use in electrophoresis and blotting are well known in the art. The desired voltage between the first and second separation phase electrodes 38, 40 is between 80 and 150 volts. The power source employs switching means for electrical isolation of said separation phase electrodes 38, 40 from said blotting transfer electrodes 50, 52.

The upper chamber 34 and lower chamber 36 are each filled with a buffer solution 56 and are electrically connected to each other via the electrically conducting gel/membrane combination unit 10, which allows negative charges to pass from the first separation electrode 38, through the buffer 56 in the upper chamber 34, through gel 6 to buffer 56 in the lower chamber 36 to the second separation electrode 40. This is accomplished in part due to the conductive polymers housing the gel having higher resistance than the gel they house. The rear panel 68 has one or more openings 74 in its lower region to allow the buffer solution 56 from the lower chamber 36 to fill up to the lower surface 18 of the gel 6 to provide an electrical connection from the second separation electrode 40 to the gel 6. The buffer solution 56 in the upper chamber 34 and lower chamber 36 may be the same buffer solution, or may be different buffer solutions, where in some embodiments, the buffer solution 56 in the upper chamber 34 may include an antioxidant.

The wires electrify the buffer solution 56 causing the solution in the upper chamber 34 to act as the cathode (−) and solution in the lower chamber 36 to act as the anode (+). Proteins in a sample buffer containing sodium dodecyl sulfate (SDS), or other buffers that are well known in the art, impart proteins with negative net charge so that when they are in the gel 6, they move from the cathode (−) 38 to the anode (+) 40 by the electromotive force (EMF) created from the power source as known in the art. By placing proteins in wells 84 in the gel and applying an electric field, the proteins will move through the gel 6 at different rates, determined largely by their mass. Thus, the first separation electrode 38 and second separation electrode 40 act as the cathode and anode, respectively, during the electrophoresis phase that separates proteins and other macromolecules by size.

The buffer solution 56 in the upper chamber 34 and lower chamber 36 are not in liquid contact with each other, but still in electrical contact with each other. The buffer solution 56 in each chamber 34, 36 is prevented from contact by the gel/membrane combination unit 10 and gaskets 44, 46 that prevent the buffer solution 56 from filling the entirety of the tank apparatus 30. A rear panel gasket 46 is disposed on the inner surface of the rear panel 68 of the tank apparatus 10. The rear panel gasket 46 prevents buffer 56 from contacting a blotting electrode 52, which would cause unwanted electrical current flow during the separation phase. Additionally, there is a lip gasket 44 disposed along the outer surface of the lip 58. The lip gasket 44 prevents buffer solution 56, necessary for the separation phase, from contacting the cooling solution 54 used in the cooling chamber 42. The cooling chamber can be filled with water, buffer, or other type of coolant. Gaskets in the preferred embodiments are made from rubber, silicone, or other materials commonly known in the art that form seals that prevents liquid seepage.

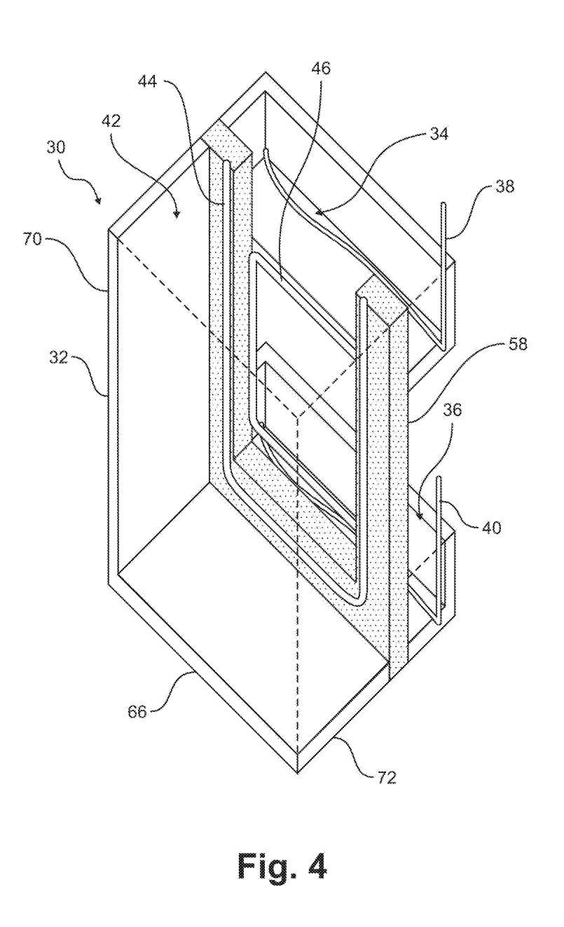

The rear panel gasket 46 is positioned so that when the precast gel/membrane combination unit 10 is placed within the tank apparatus 30, the outer surface 24 of the second conductive plate 4 contacts and is pressed against the gasket 46. The lip gasket 44 is positioned so that the inner surface of the first conductive plate 2 is pressed against the lip gasket 44. As shown in FIG. 4, the rear panel gasket 46 is a continuous loop along the inner surface of the rear panel 68. The lip gasket 44 is an open shape having a bottom region connected to two side regions (with no top region to form a loop gasket). The lack of a rubber sealing structure on the top allows the buffer 56 to be in electrical contact with the top 20 of gel 6. In sum, the gaskets 44, 46 are positioned such that when the precast gel/membrane combination unit 10 is placed correctly within the tank apparatus 10, the gaskets 44, 46 form seals that keep the upper chamber 34 and lower chambers 36 (necessary for the electrophoresis phase) separate from the cooling chamber 42 and other structures required during the protein transfer phase. Another feature to prevent the chambers 34, 36, 42 from being in liquid and/or electrical contact with each other is that the first conductive plate 2 is larger than the second conductive plate 4. In a preferred embodiment, the first conductive plate is approximately 12 cm×12 cm and the second conductive plate 4 is approximately 10 cm×10 cm (approximately the same dimensions of the gel 6). The larger first conductive plate 2 allows for the first conductive plate 2 to contact the lip gasket 44 and the smaller second conductive plate 4 to be in contact with the rear panel gasket 46. The smaller second conductive plate 4 allows the buffer solution 56 to pass over and under the second conductive plate 4 in the upper chamber 34 and lower chamber 36, respectively, to reach the gel 6, but not pass by the larger first conductive plate 2. This prevents the buffer solution 56 from entering into the cooling chamber 42 and contacting the transfer electrodes 50, 52 used in the transfer/blotting phase.

After the separation phase, where the proteins have been separated vertically along the gel 6 due to the electric field created by the upper and lower separation electrodes 38, 40, the electrical current is then shifted from the separation electrodes 38, 40 to transfer electrodes 50, 52 for use in the transfer/blotting phase, which forces the proteins to move from the gel 6 to the membrane 12 via the EMF supplied by the transfer electrodes 50, 52 to the first conductive plate 2 and second conductive plate 4. The first conductive plate 2 is in electrical contact with a first transfer electrode 50 connected to a power source and the second conductive plate 4 is in electrical contact with a second transfer electrode 52. Since the first and second conductive plates 2, 4 are made from conductive plastics, when current is applied to the transfer electrodes 50, 52, the conductive plates 2, 4 act as plate electrodes.

In the embodiment shown in FIG. 3, the first transfer electrode 50 is an arc shaped metal brace to provide sufficient tension to hold the precast gel/membrane combination unit 10 in place against the gaskets 44, 46 to form the different chambers 34, 36, 42. The transfer electrode 50, may also be a separate element from the structure that braces/tensions the precast gel/membrane combination unit 10 inside the tank 30, against the various gaskets 44, 46 within the tank 30. In other embodiments, the first transfer electrode 50 may be separated into two or more brackets (e.g. one on the left side, one on the right side) so that the user can still view the gel 6, but that the precast gel/membrane combination unit 10 would still be held firmly in place. Other types of braces/brackets/tensioners could also be placed inside the cooling chamber 42 without departing from the spirit of the invention as long as there is tension and electrical contact from the transfer electrode 50 to the first conductive plate.

The second transfer electrode 52 is disposed along the inner surface of the rear panel 68 and acts as the anode (+) during the protein transfer/blotting mode. In the embodiment shown in FIG. 3, the second transfer electrode 52 has a spring or recoil action so that the transfer electrode 52 makes sufficient contact with the second conductive plate 4. In other embodiments, the transfer electrode 52 may be a separate element from an element having the spring or recoil action to help brace the precast gel/membrane combination unit 10 inside the tank 30 against an opposing bracing member. An electrical power source connects the first transfer electrode 50 with the second transfer electrode 52 and is applied to the transfer electrodes 50, 52 such that the first conductive plate 2 acts as the cathode (−) and second conductive plate acts as the anode (+) during the transfer/blotting phase. The electrical source shall provide enough electricity to achieve a voltage difference between the two plate electrodes to achieve sufficient transfer of the proteins toward the second conductive plate 4 to the transfer membrane 12 housed within the precast gel/membrane combination unit 10. A typical voltage applied during the blotting mode is around 30 volts.

FIG. 4 is a perspective view of the tank apparatus 30 without the precast gel/membrane combination unit 10. The precast gel/membrane combination unit 10 is placed adjacent to (on the left side) of the lip 58 having the lip gasket 44. Since the first conductive plate 2 is larger than the second conductive plate 4, the first conductive plate 2 lays on the outer surface, while the electrophoresis gel 6, low conductivity gel 8, transfer membrane 12 and filter paper 60 are within the inner cavity of the lip 58 and the second conductive plate 4 is pressed against the rear panel gasket 46.

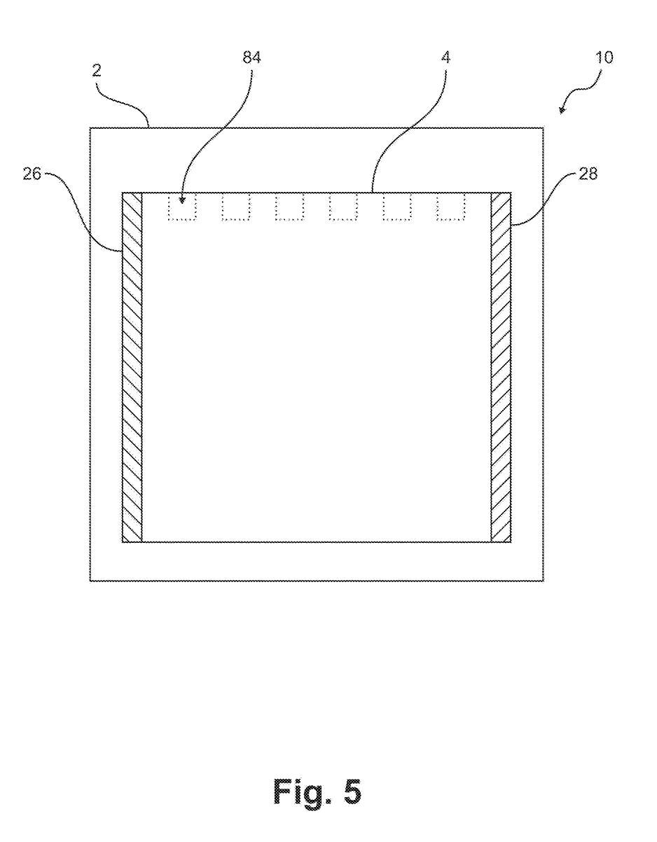

FIG. 5 is a front view of the precast gel/membrane combination unit 10. As described earlier, the first conductive plate 2 is larger than the second conductive plate 4. The gel 6, low conductivity gel 8, transfer membrane 12, and filter paper 60 (not seen in FIG. 5, as they are blocked by the second conductive plate 4), are all sandwiched between the first and second conductive plates 2, 4. In the embodiment of FIG. 5, the gel 6, low conductivity gel 8, transfer membrane 12, and filter paper 60 have approximately the same height, but may have a slightly smaller width to allow for the insertion of the insulative plastic strips 26, 28, that flank the sides of the gel 6. Wells 84 within the gel 6 may be created by a gel comb during formation of the gel where proteins can be deposited.

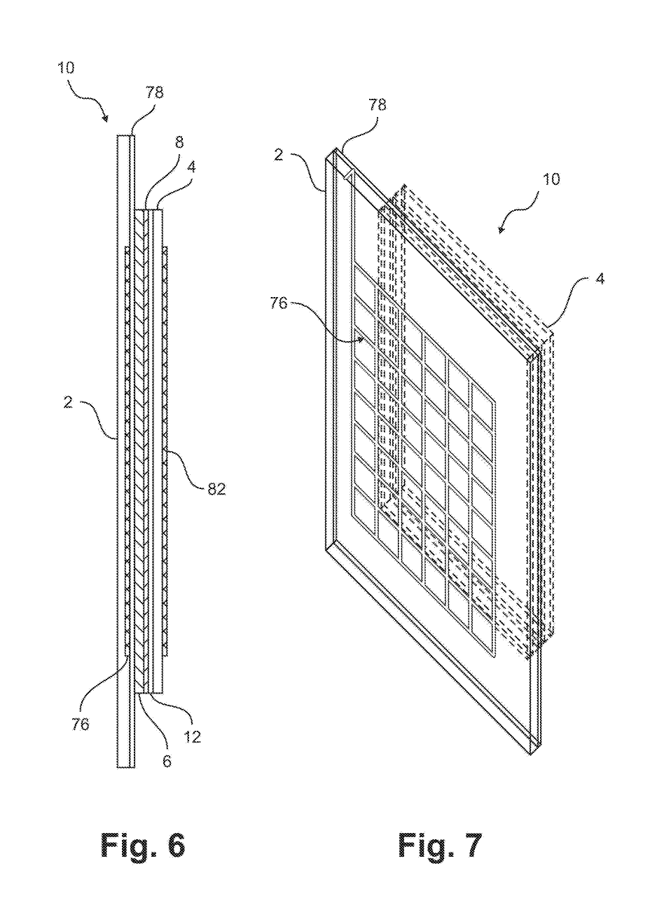

FIGS. 6-7 show another embodiment of a precast gel/membrane combination unit 10. Instead of the first plate 2 being made entirely from a conductive polymer, the first plate is made from a clear plastic having static dissipative properties, with volume resistivity in the range of approximately 10⁸ to 10¹⁰ ohm-cm. On the inside surface of the first plate 2 or embedded within the first plate 2 are a plurality of conductive wires or mesh 76, which may be arranged in a grid or array. The mesh 76 distributes electric current along the inner surface of the first plate 2. Also disposed along the inner surface of the first plate 2, and in electrical contact with the mesh 76 is a thin transparent conductive layer 78, which may be made from a thin transparent conductive polymer or transparent conducting film (TCF) 78 having a volume resistivity in the range of approximately 10⁴ to 10⁵ ohm-cm. TCFs are known in the art, and in the embodiments of FIGS. 6-7, the first plate 2 is coated with the TCF film 76 or layer of transparent conductive polymer, thereby forming a plate electrode. The wire mesh 76 ensures that electric charge is evenly spread along the film 78 or thin transparent conductive polymer. The TCF may be a conductive polymer but can also be a transparent conducting metal, including, but not limited to indium tin oxide (ITO), fluorine doped tin (FTO), doped zinc oxide, aluminum-doped zinc-oxide (AZO). In a preferred embodiment, the TCF is ITO, as it is chemically resistant to moisture, which is advantageous for long-term storage of the precast gel/membrane combination unit 10. One possible advantage of a system using TCFs of a thin coat of a transparent conductive polymer is that the transparency of a conductive polymer is reduced as thickness increases, even among known transparent conductive polymers. By limiting the conductive region of the first rigid conductive plate 2 to a thin region of the plate (or layered on top of the plate 2), the gel/membrane combination unit 10 maintains high transparency and high rigidity to form the structural support of an electrophoresis gel 6.

FIGS. 6-7 shows the side view and perspective view, respectively, of the first plate 2, wire mesh 76, TCF or thin transparent conductive polymer 78, electrophoresis gel 6, transfer membrane 12, second plate 4, and an additional wire mesh 76 disposed on the outside surface 24 of the second plate 4. The wire mesh 76 disposed along the outside surface of the second 4 efficiently distributes electrical current along the rear plate 4 to more efficiently transfer macromolecules from the gel 6 to the transfer membrane 12.

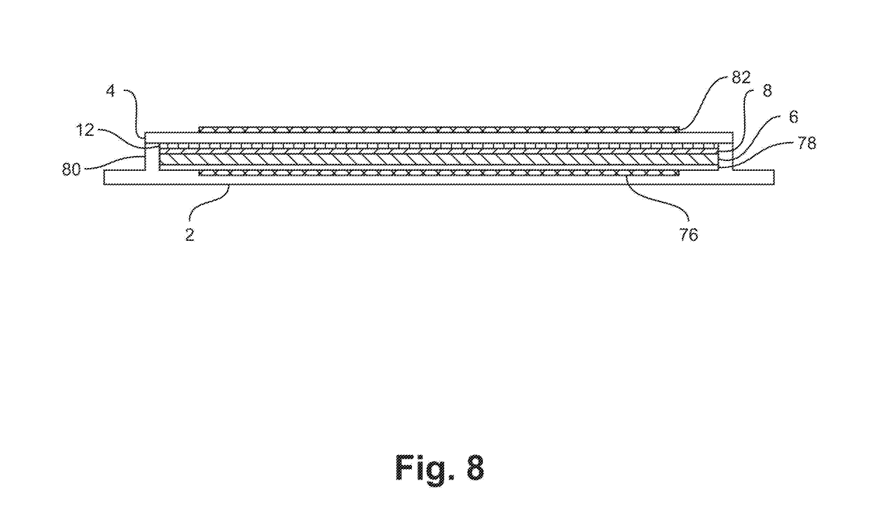

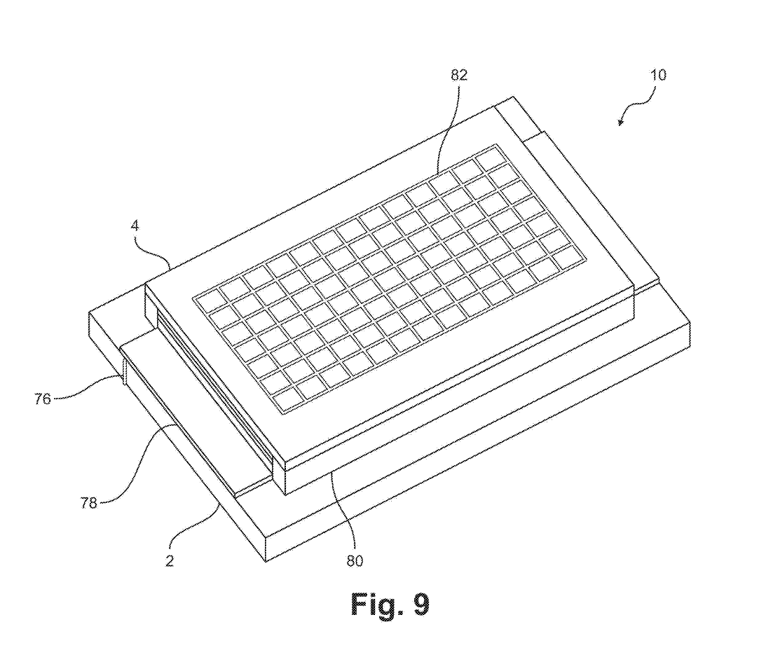

FIGS. 8-9 illustrate another example of the precast gel/membrane combination unit 10. FIG. 8 is a top view and FIG. 9 is a perspective view of the precast gel/membrane combination unit 10. This embodiment includes two pedestal projections 80 of the non-conducting static dissipative front plate 2 abutting the inner surface of the rear plate 4. The second plate 4 rests over the pedestals 80, thereby forming a gap between the inner surface of the first plate 2 and the inner surface of the second plate 16. The gap may vary depending on the thickness of the gel. In one embodiment, the gap will range from about 0.1 cm to about 0.5 cm. The gap holds the various components needed for proper separation and blotting of macromolecules, as previously described, such as the electrophoresis gel 6, low conductivity gel 8, transfer membrane 12, and filter paper 60. In this embodiment, the entirety of the first plate 2 is transparent but not highly conductive. Conductivity along the inner surface of the first plate is accomplished through the use of a thin conductive polymer layer or other type of transparent conductive film 78, which overlays or is connected to a first wire mesh 76 that does not visually obstruct the view of gel 6 through the first plate 2. The first wire mesh 76 distributes the electric charge substantially evenly along the entirety of the conductive polymer layer 78 so that an electric field is produced on the thin conductive polymer layer 78, which then acts as a plate electrode. In a preferred embodiment, the film or thin transparent polymer has a thickness of less than 1 mm and has a volume resistivity between 10⁴ and 10⁵ ohm-com. The rear plate 4 has a second wire mesh 82, which creates a substantially even charge along the entirety of the rear plate 4, thereby producing a substantially even electrical field so that macromolecules are efficiently and evenly transferred from the gel 6 to the transfer membrane 12 during the blotting phase.

In the preferred embodiment, the separation/transfer buffer 56 will have a volume resistivity of approximately 10-200 ohm-cm. The conductive plastic front plate 2 and rear plate 4 will have volume resistivities in the range of 10³ to 10⁵ ohm-cm. In the case of a thin conductive coating or film 78 on the inner surface of the front plate 2, the volume resistivity of the coating or film will be in the range of 10⁴ to 10⁵ ohm-cm, and the front plate 2 will be made of a static-dissipative transparent plastic with a volume resistivity of 10⁸ to 10¹⁰ ohm-cm. These ranges will allow for the electric current to flow through the gel during the separation phase when a power source is applied to separation electrodes 38, 40 rather than through the front and rear plates 2, 4. Then, when the power source is applied to 50 and 52, or to 76 and 52, the electric current will flow substantially perpendicular to the length of the gel from the front plate 2, through the gels 6, 8 and membrane 12, to the rear plate 4, allowing for the proteins to be embedded on the blotting membrane 12 during the blotting phase. While these ranges have been described in terms of exemplary embodiments, it is to be understood that they are not limiting, whereas any embodiment in which the buffer 56 and gel 6 have a reasonably lower resistivity than the conductive polymers (i.e. plates 2, 4) that house them, and conversely that the conductive polymers (i.e. plates 2, 4) have a reasonably higher conductivity than the buffer 56 and gel 6 will allow for the described separation and transfer phases.

While the invention has been described in terms of exemplary embodiments, it is to be understood that the words which have been used herein are words of description and not of limitation. As is understood by persons of ordinary skill in the art, a variety of modifications can be made without departing from the scope of the invention defined by the following claims, which should be given their fullest, fair scope.

1. An apparatus for electrophoretic separation and blotting, comprising:
a first electrically semi-conductive plate made from a transparent semi-conductive polymer;
a second electrically conductive plate made from a semi-conductive polymer, the second electrically semi-conductive plate substantially parallel to the first electrically semi-conductive plate;
an electrophoresis gel; and,
a blotting membrane;
wherein the electrophoresis gel is between the first electrically semi-conductive plate and the blotting membrane; and
wherein the blotting membrane is between the electrophoresis gel and the second electrically semi-conductive plate.

2. The apparatus of claim 1, further comprising:
a low conductivity gel having a lower conductivity than the electrophoresis gel, wherein the low conductivity gel is between the electrophoresis gel and the blotting membrane, whereby the low conductivity gel prevents migration of macromolecules from diffusing away from the electrophoresis gel and adhering to the blotting membrane during a macromolecule separation phase;
a filter paper between the second semi-conductive plate and the blotting membrane, whereby the filter paper, when wet, acts as an ion reservoir and provides substantial electrical contact between the blotting membrane and the second semi-conductive plate to aid in transferring macromolecules from the electrophoresis gel through the low conductivity gel to the blotting membrane;
wherein the blotting membrane is at least one of a nitrocellulose membrane, polyvinylidene difluoride (PVDF) membrane, or nylon membrane.

3. The apparatus of claim 1, further comprising electrically conducting wires disposed on or within the first electrically semi-conductive plate, wherein the first electrically semi-conductive plate is characterized as having an outer electrically conductive transparent layer overlaying a transparent plastic having static dissipative properties.

4. The apparatus of claim 3, wherein the outer electrically semi-conductive transparent layer disposed on an inner surface of the first plate, wherein the layer comprises at least one of indium tin oxide, fluorine doped tin, doped zinc oxide, and aluminum-doped zinc-oxide.

5. The apparatus of claim 4, wherein the transparent semi-conductive layer has a thickness of less than 1 mm.

6. The apparatus of claim 1, wherein the first electrically conductive plate is made from a polymer containing one or more types of transparent semi-conducting polymers selected from the group consisting of polyacetylene, poly(pyrrole)s, polyanilines, polythiophene, poly(3,4-ethylenedioxythiophene), poly(p-phenylene) sulfide, poly(p-phenylene vinylene), and their derivatives.

7. The apparatus of claim 1, wherein the first electrically semi-conductive plate and the second electrically conductive plate are substantially rigid and designed to structurally support a slab gel.

8. An apparatus for electrophoretic separation and blotting macromolecules, comprising:
a first plate made of a transparent polymer;
an electrically semi-conductive transparent layer adjacent to the first plate;
a second plate substantially parallel to the first plate;
an electrophoresis gel; and,
a blotting membrane;
wherein the electrophoresis gel is between the electrically semi-conductive transparent layer and the blotting membrane; and,
wherein the blotting membrane is between the electrophoresis gel and the second plate.

9. The apparatus of claim 8, further comprising:
a first electrically conductive wire array in contact with first plate and the electrically semi-conductive transparent layer; and,
whereby the first electrically conductive wire array distributes charge along the electrically conductive transparent layer.

10. The apparatus of claim 9, wherein the electrically semi-conductive transparent layer is an electrically conductive film disposed on an inner surface of the first plate, wherein the electrically semi-conductive film is made of a film containing one or more types of conductive transparent metals selected from the group consisting of indium tin oxide, fluorine doped tin, doped zinc oxide, aluminum-doped zinc-oxide, and their derivatives.

11. The apparatus of claim 8, wherein the first electrically semi-conductive plate is made from a polymer containing one or more types of transparent semi-conducting polymers selected from the group consisting of polyacetylene, poly(pyrrole)s, polyanilines, polythiophene, poly(3,4-ethylenedioxythiophene), poly(p-phenylene) sulfide, poly(p-phenylene vinylene), and their derivatives.

12. The apparatus of claim 8, wherein the transparent semi-conductive layer has a thickness of less than 1 mm.

13. The apparatus of claim 8, wherein the transparent semi-conductive layer has a volume resistivity between 10⁴ and 10⁵ ohm-cm and the first plate has a volume resistivity between 10⁸ and 10¹⁰ ohm-cm.

14. The apparatus of claim 8, further comprising a second electrically conducting wire array adjacent to the second plate, whereby the second electrically conducting wire array distributes electrical charge along the second plate.

15. A system for both electrophoretic separation and blotting macromolecules, comprising:
a liquid receptacle tank having an upper buffer chamber and a lower buffer chamber, a front panel, rear panel, and bottom;
a first separation phase electrode in the upper buffer chamber;
a second separation phase electrode in the lower buffer chamber;
a first blotting phase electrode;
a second blotting phase electrode;
a precast gel/membrane combination unit having (i) a first electrically semi-conductive plate made from a transparent conductive polymer, (ii) a second electrically semi-conductive plate made from a conductive polymer, the second electrically semi-conductive plate substantially parallel to the first electrically semi-conductive plate, (iii) an electrophoresis gel, and (iv) a blotting membrane; and,
a power supply, wherein the power supply is configured to apply a voltage to the first separation phase electrode and second separation phase electrode to perform electrophoretic separation of macromolecules along the electrophoresis gel, and wherein the power supply is configured to automatically switch a voltage from the first and second separation phase electrodes to the first and second blotting phase electrodes, whereby switching the voltage allows a user to perform electrophoretic separation of proteins and transfer of proteins onto the blotting membrane.

16. The system of claim 15,
wherein the liquid receptacle tank further comprises a cooling chamber housing the first blotting phase electrode; and,
a gasket disposed on the rear panel of the liquid tank receptacle, whereby the gasket prevents liquid from flowing from the upper chamber to the lower chamber when the precast gel/membrane combination unit is placed within the liquid receptacle tank.

17. A method for separation and post-separation transfer of macromolecules to a blotting membrane, the method comprising the steps of:
providing an apparatus in a first orientation within a liquid receptacle tank, wherein the apparatus has a first electrically conductive plate composed of (i) a transparent semi-conductive polymer, (ii) second electrically semi-conductive plate made of a semi-conductive polymer, the second electrically semi-conductive plate substantially parallel to the first electrically semi-conductive plate, (iii) an electrophoresis gel, and (iv) a blotting membrane, wherein the electrophoresis gel is between the first semi-conductive plate and the blotting membrane, and wherein the blotting membrane is between the electrophoresis gel and the second electrically semi-conductive plate;
separating macromolecules along the gel of the apparatus by applying a first electrical driving force to a pair of separation electrodes, wherein separating macromolecules along the gel occurs in the first orientation of the apparatus; and
discontinuing the first electrical driving force to the pair of separation electrodes;
transferring macromolecules through the gel to the blotting membrane by applying a second electrical driving force substantially perpendicular to the first electrical driving force while maintaining the first orientation of the apparatus, thereby combining the steps of electrophoresis and transfer in a single liquid receptacle tank without having to reorient the apparatus between the separating step and transferring step.

18. The method of claim 17 further comprising the step of:
pre-programming a power source automatically to apply the first electrical driving force, discontinuing the first electrical driving force, and applying the second electrical driving force substantially perpendicular to the first electrical driving force.

19. The apparatus of claim 1, wherein the transparent semi-conductive polymer has a volume resistivity between 10³ -10⁸ ohm-cm.

20. The apparatus of claim 1, wherein the transparent semi-conductive polymer has a volume resistivity between 10³ -10⁵ ohm-cm.
